(19) United States
(12) Patent Application Publication
(10) Pub. No.: US 2026/0306857 A1
(43) Pub. Date: Oct. 8, 2026

(54) SYSTEMS, METHODS, AND DEVICES FOR PROVIDING STIMULATION THERAPY USING DISPLAY OF SENSED PHYSIOLOGICAL SIGNALS

(71) Applicant: Medtronic, Inc., Minneapolis, MN (US)

(72) Inventors: Scott R. Stanslaski, Shoreview, MN (US); Thomas L. Chouinard, Maple Grove, MN (US); Todd D. Zenisek, Georgetown, TX (US); Andrew H. Houchins, Hugo, MN (US); Nicholas R. Unger, Salt Lake City, UT (US); Stephanie L. Szolwinski, Anoka, MN (US); Erik J. Peterson, Fridley, MN (US); Steven J. Stroncek, Edina, MN (US); Steven M. Goetz, North Oaks, MN (US)

(21) Appl. No.: 19/479,078

(22) PCT Filed: Apr. 3, 2024
(86) PCT No.: PCT/IB2024/053243

§ 371 Date: Oct. 27, 2025

Related U.S. Application Data
(60) Provisional application No. 63/462,327, filed on Apr. 27, 2023.

(51) Int. Cl. A61N 1/372; A61N 1/36; G06F 3/04847

(52) U.S. Cl. CPC A61N 1/37247; A61N 1/36135; A61N 1/3615; A61N 1/3606; G06F 3/04847

(57) ABSTRACT

Physiological signals, such as local field potential signals, are sensed by an implantable medical device and are displayed for viewing by a clinician during aspects of providing stimulation therapy. In one instance, local field potential signals may be displayed that have been sensed during an auto-capture of thresholds to be used for stimulation therapy, such as deep brain stimulation, with the auto-captured thresholds displayed together with the physiological signals. In another instance, local field potential signals are displayed together with stimulation signals so that the relationship of the local field potential signals and the stimulation signals is visible, and this display of these signals may be done in real-time as the implantable medical device is capturing the signals and transmitting them to the external device.

This Application claims priority from U.S. Provisional Patent Application 63/462,327, filed 27 Apr. 2023, the entire content of which is incorporated herein by reference.

### TECHNICAL FIELD

Embodiments relate to providing stimulation therapy. More particularly, embodiments relate to systems, methods, and devices that provide stimulation therapy using display of sensed physiological signals.

### BACKGROUND

Patients suffering from certain medical conditions may benefit from electrical stimulation therapy. A medical system, such as an externally located or implanted implantable medical device coupled to an implantable lead, provides this electrical stimulation therapy to a target site within the body of the patient. For instance, certain neurological conditions may benefit from neurostimulation therapy being provided to neural tissue in the brain, the spine, or elsewhere in the body. The implantable medical lead extending from the implantable medical device can route the electrical stimulation signals to the target site as well as allow the medical device to sense physiological signals occurring at the target site.

Certain types of stimulation therapy can be more effective by controlling the stimulation therapy based on how the body of the patient is responding to the stimulation therapy. For instance, in deep brain stimulation therapy, it can be beneficial to use an adaptive mode that senses for local field potential signals contemporaneously with providing a stimulation signal that includes electrical pulses, and a stimulation signal characteristic such as the amplitude of the electrical stimulation pulses may be adjusted based upon the power present in those local field potential signals. Typically, in adaptive deep brain stimulation, a minimum amplitude and a maximum amplitude of the stimulation signal along with one or more thresholds of the physiological signal are set for the patient, and the strength of the local field potential being sensed is compared to the one or more thresholds. If a particular threshold is exceeded in the direction of interest, such as being stronger than an upper threshold or weaker than a lower threshold, then the stimulation characteristic may be altered in the way necessary to alter the local field potential as desired. For instance, if the local field potential exceeds an upper threshold by having a greater electrical power than the upper threshold, then the stimulation current amplitude may be increased toward the maximum stimulation amplitude. Likewise, if the local field potential exceeds a lower threshold by having a lesser electrical power than the lower threshold, then the stimulation current amplitude may be decreased toward the minimum stimulation amplitude.

An implantable medical device providing the adaptive deep brain stimulation therapy may have the ability to automatically determine parameters including upper and lower thresholds of the local field potential signal power within a range of minimum and maximum stimulation current amplitudes by employing an auto-capture algorithm during an auto-capture period. While this approach to adaptive therapy can be beneficial, issues can arise. For instance, various factors can result in an ineffective auto-capture where thresholds for a given range of stimulation current are not optimal. Furthermore, even where an initial auto-capture may produce successful therapy, over time the auto-captured values of thresholds and/or the stimulation current amplitude range are no longer optimal for the patient. For instance, the disease necessitating the deep brain stimulation may progress over time making the upper and lower thresholds and/or the stimulation current amplitude range result in ineffective therapy. As another example, a change in the medication state of the patient may render the upper and lower thresholds and/or the stimulation current amplitude range inappropriate for producing effective stimulation therapy.

### SUMMARY

Embodiments disclosed herein address issues such as these and others by providing stimulation therapy by producing a display of the sensed physiological signal such as a local field potential signal. The local field potential may be displayed in relation to auto-captured thresholds to provide a visual indication of the success of the auto-capture function to establish effective thresholds. Additionally, embodiments may provide for a real-time display of both the physiological signal, such as a local field potential, that is being sensed together with the stimulation therapy signal that is evoking such physiological signals and the thresholds being used to provide the stimulation therapy signal to further provide a visual indication of the efficacy of the stimulation therapy being provided. Furthermore, controls may be provided to allow the thresholds and/or the stimulation amplitude range from the auto-capture to be modified and/or to allow the thresholds and/or stimulation amplitude range to be modified in real-time during application of the stimulation therapy that utilizes the thresholds and stimulation amplitude range.

Embodiments provide systems, devices, and methods to provide adaptive neurostimulation therapy from an implantable neurostimulation device by use of an external device. This includes sending an instruction by the external device to the implantable neurostimulation device to perform an automatic threshold capture for providing neurostimulation therapy while sensing a local field potential signal. This also includes receiving at the external device data representative of the sensed local field potential signal occurring during automatic threshold capture from the implantable neurostimulation device and displaying the sensed local field potential signal at the external device.

Embodiments provide systems, devices, and methods to provide adaptive neurostimulation therapy from an implantable neurostimulation device by use of an external device. This includes sending an instruction by the external device to the implantable neurostimulation device to provide adaptive stimulation therapy by applying a stimulation signal within a range of first and second amplitudes while sensing a local field potential signal and comparing the local field potential signal to at least one threshold. This includes receiving at the external device streaming data representative of the sensed local field potential signal from the implantable neurostimulation device and displaying the sensed local field potential signal represented by the streaming data at the external device. This also includes displaying at least one threshold with the sensed local field potential signal and displaying at least one threshold control. Upon receiving a manipulation of at least one threshold control by a user of the external device, this further includes displaying at the external device at least one resulting threshold with the sensed field potential and sending by the external device an instruction to the implantable neurological device to implement the resulting at least one threshold in real-time while providing the adaptive stimulation therapy.

Embodiments provide systems, devices, and methods to provide adaptive neurostimulation therapy from an implantable neurostimulation device by use of an external device. This includes sending an instruction by the external device to the implantable neurostimulation device to provide adaptive stimulation therapy by applying a stimulation signal within a range of first and second amplitudes while sensing a local field potential signal and comparing the local field potential signal to at least one threshold. This includes receiving at the external device streaming data representative of the sensed local field potential signal from the implantable neurostimulation device and displaying the sensed local field potential represented by the streaming data at the external device. This also includes receiving at the external device streaming data representative of the stimulation signal from the implantable neurostimulation device and displaying by the external device the stimulation signal adjacent the sensed local field potential signal at the external device along a common time axis. This further includes providing by the external device a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

### DESCRIPTION OF THE DRAWINGS

FIG. 1 shows an example of an implantable medical system of a patient.

FIG. 2 shows an example of an implantable medical system where an external device is present to communicate with the implantable medical device and generate displays of information.

FIG. 3 shows an example of the components of an external device that communicates with the implantable medical device and generates the displays of information.

FIG. 4 shows an example of the components of an implantable medical device.

FIG. 5 shows a first example of a display provided by an external device to show local field potential signal data sensed by the implantable medical device during the auto-capture process and related thresholds related to the auto-capture process.

FIG. 6 shows a second example of a display provided by an external device to show a real-time or paused local field potential signal and a corresponding stimulation signal being provided by the implantable medical device.

FIG. 7 shows a third example of a display provided by an external device to show a real-time or paused local field potential signal and a corresponding stimulation signal being provided by the implantable medical device and to receive and show annotations added by a user.

FIG. 8 shows an example of an operational flow performed by the external device when providing the display of FIG. 5.

FIG. 9 shows an example of an operational flow performed by the external device when providing the display of FIGS. 6 and 7.

### DETAILED DESCRIPTION

Embodiments provide for the display of information related to the application of physiological thresholds to control stimulation therapy such as adaptive deep brain stimulation for a patient. Embodiments also provide for the display of controls related to manually adjusting aspects of providing the stimulation therapy including physiological thresholds and related stimulation amplitude limits. These displays of information allow a clinician to review the relationships of the physiological thresholds, the stimulation amplitude limits, and the resulting physiological signals.

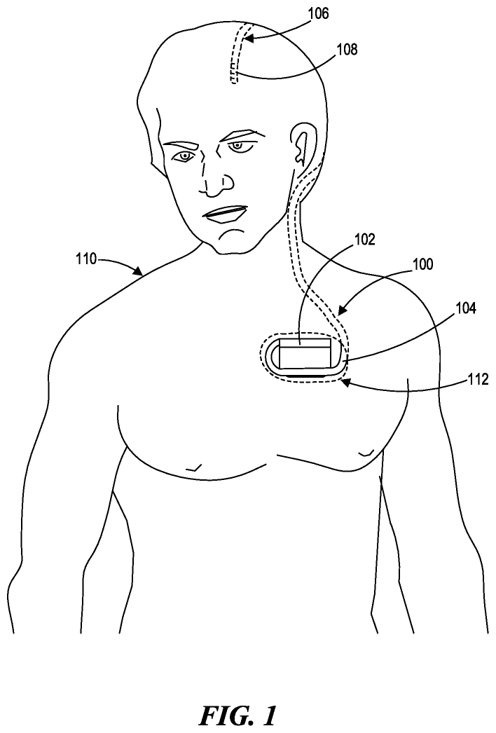

FIG. 1 shows a typical environment for an implantable medical system 100. In this example, the implantable medical system 100 is present within a body 110 of a patient. The implantable medical system 100 of this example includes an implantable stimulation device 102 that has been implanted into a subcutaneous or subfascial pocket 112. At least one implantable medical lead 104, or lead and extension combination, is routed between a target stimulation site and the implantable stimulation device 102.

In this example, the implantable stimulation device 102 is a neurostimulator configured for adaptive deep brain stimulation, as the lead 104 has a distal end 106 and distal electrodes 108 positioned within the brain at the target location for the stimulation to be applied. Other examples of implantable medical devices include but are not limited to neurostimulators for spinal cord stimulation where the lead 104 is directed to the spinal cord, peripheral nerve stimulation where the lead 104 is directed to a peripheral nerve, pelvic nerve stimulation where the lead 104 is directed to a pelvic nerve, cardiac stimulation where the lead 104 is directed to cardiac tissue, and the like.

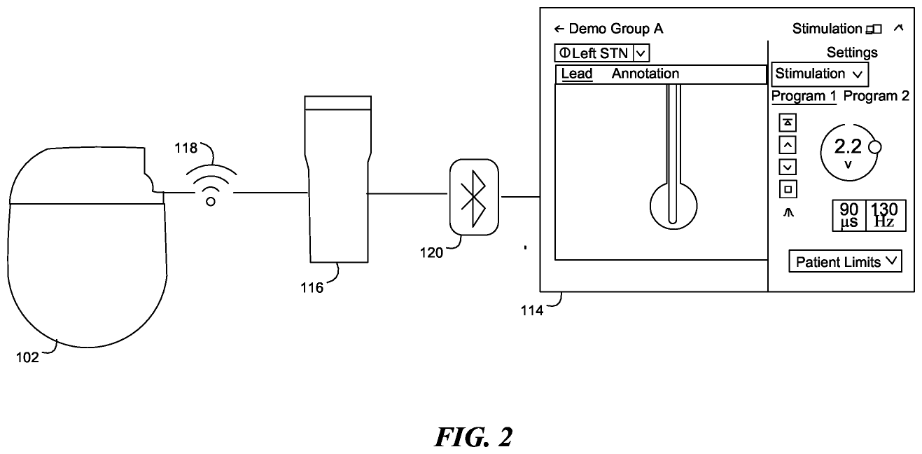

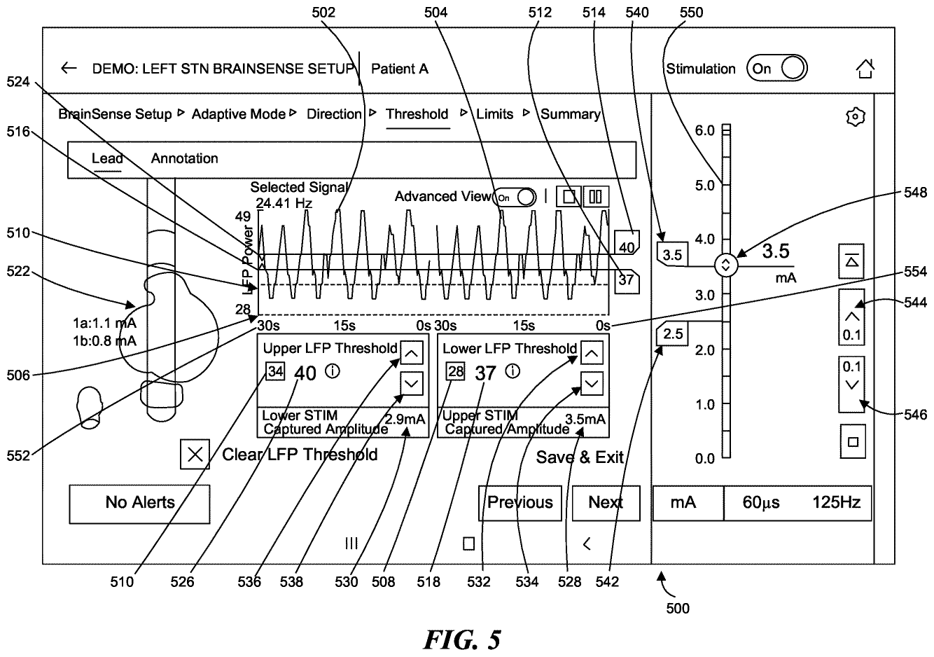

FIG. 2 shows an external device 114 such as a hand-held tablet, personal computer, or similar device producing a display of information for a clinician or other user while communicating with the implantable medical device 102. As discussed in more detail below with respect to FIGS. 5-9, the external device 114 may display information obtained from the implantable medical device 102, including information being streamed from the implantable medical device in real-time, to produce a display of information that a clinician may use to adjust parameters of the stimulation therapy being applied by the implantable medical device 102.

In the example of FIG. 2 and is well-known in the art, the external device 114 relies upon an intermediary communication device 116 that is also external of the patient. In the example shown, the external device 114 may communicate using wired or wireless signals over a communication link 120, such as wireless signals using the Bluetooth® protocol of the Bluetooth Special Interest Group, or other short to medium range wireless signals. The communicator device 116 may then relay information to the implantable medical device 102 using wireless signals 118. The wireless signals 118 may be proximity signals such as those that rely upon near field inductive coupling, may be arms-length wireless signaling such as using the Medical Implant Communication System (MICS), may be a longer-range form of wireless signaling, and so forth. Furthermore, in other examples, the implantable medical device 102 and the external device 114 may utilize the same wireless communication type, such as where both devices utilize MICS, to communicate directly and thereby eliminate the use of the intermediary communicator device 116.

Regardless of the type of communication path between the external device 114 and the implantable medical device 102, the external device 114 may send information to the implantable medical device 102 and receive information from the implantable medical device 102, including exchanging information in real-time while the implantable medical device 102 is providing stimulation therapy to the patient. As discussed below, this allows the user of the external device 114 to monitor the efficacy of any auto-threshold capture being performed by the implantable medical device 102 when configuring stimulation therapy and also allows the user to make real-time adjustments to parameters of the stimulation therapy such as the physiological thresholds used to control the stimulation signal as well as the limits of the stimulation amplitude.

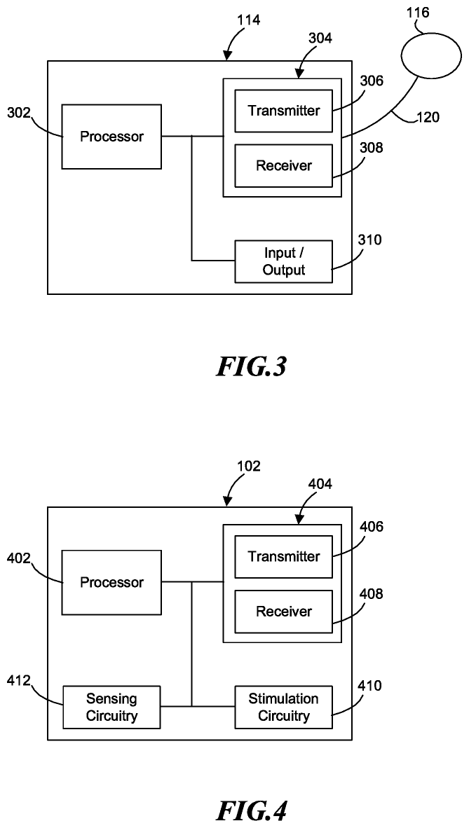

FIG. 3 shows an example of the components of the external device 114. As discussed above, the external device 114 may take various forms, such as a handheld tablet, a personal computer, and the like. The several components of the external device 114 include a processor 302 as well as a communication circuitry 304 and an input/output circuitry 310. The processor 302 interacts with the communication circuitry 304 and input/output circuitry 310 to provide the operations of the implantable medical device 102. A power supply, not shown in FIG. 2, such as a battery or a utility power interface may also be included to provide electrical power to the various components.

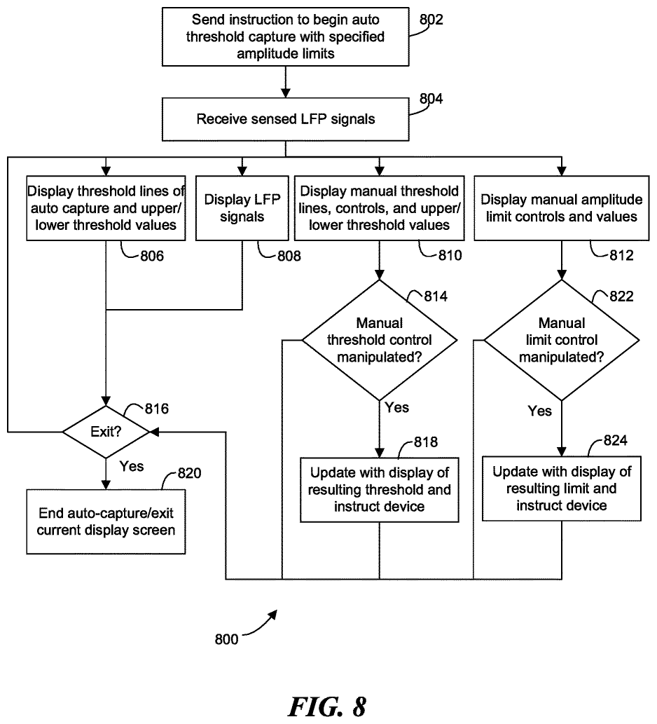

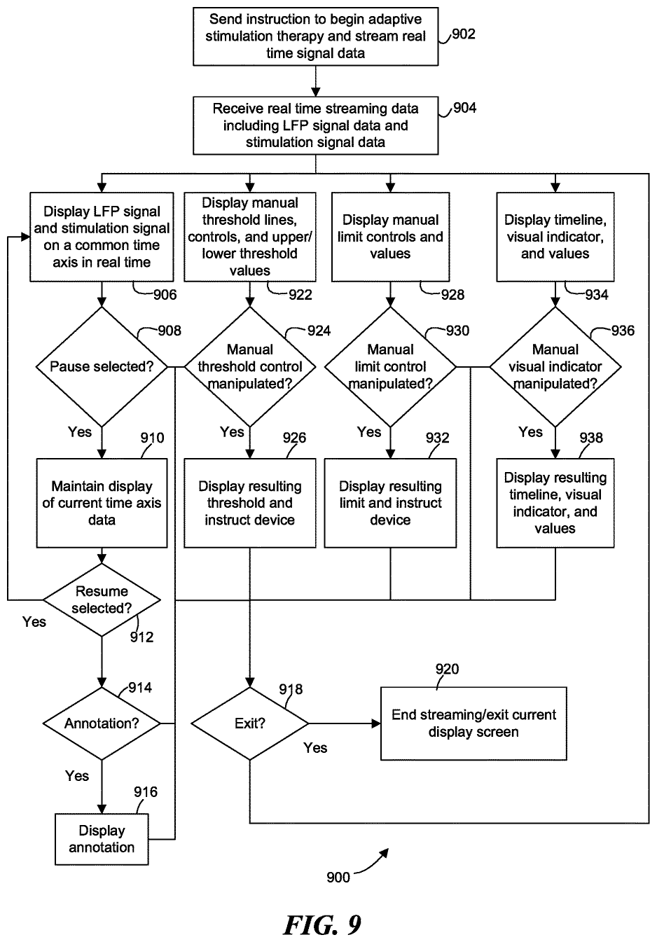

The processor 302 performs various logical operations when interacting with the other components. These operations may involve utilizing the communication circuitry 304 to exchange data with the implantable medical device 102 and to produce relevant displays of information and receive relevant input from a user viewing the displays. Examples of these logical operations are discussed below in relation to FIGS. 8 and 9. The processor 302 may be of various forms such as a general purpose programmable processor, application specific processor, hardwired digital logic, and/or various combinations. The processor 302 may utilize operational memory that is internal, external (not shown), or a combination of the two and may also utilize a storage device to retain data and programming in a long-term, non-volatile fashion.

The communication circuitry 304 includes both a transmitter circuit 306 and a receiver circuit 308 for sending and receiving wireless signals. The communication circuitry 304 is either wirelessly tethered or tethered by wire to the intermediary device 116 over the communication link 120. As previously discussed, the communication link may utilize a wireless protocol such as the Bluetooth® protocol. Alternatively, the communication link 120 may be wired and rely upon telemetry where the intermediary device 116 is a telemetry head held in very close proximity to the implantable medical device 102. As another alternative, any link with a sufficiently short latency to allow the clinician to react to changes in the patient may also be used, including remote/internet connected management.

The input/output circuitry 310 allows the external device 114 to interact with users or other devices. The input/output circuitry 310 may provide outputs such as a visual display on a screen, audio, and the like. The input/output circuitry 310 may provide inputs such as a keyboard or keypad, a mouse and/or touch screen, and the like. The input/output circuitry 310 allows users to enter information such as programming details, stimulation parameters, and other information to be provided from the external device 114 to the implantable medical device 102 as well as review information such as physiological data sent from the implantable medical device 102 to the external device 114.

FIG. 4 shows an example of the implantable medical device 102. The components of the implantable medical device 102 are contained within a housing that isolates and protects the components from the surrounding environment. Typically, the housing is a biocompatible material that forms a hermetically sealed container. The components of the implantable medical device 102 include a processor 402 as well as a communication circuitry 404. A stimulation circuitry 410 is also present to generate the stimulation signals used to provide the stimulation therapy, and sensing circuitry 412 is present to sense the physiological signals relevant to the stimulation therapy, such as the local field potential signals. The processor 402 interacts with the communication circuitry 404, stimulation circuitry 410, and sensing circuitry 412 to provide the operations of the implantable medical device 102. Although not shown, the implantable medical device 102 also includes a power source, such as an on-board battery, to provide electrical power to these components.

The processor 402 performs various logical operations when interacting with the other components. As the second device 404 may also measure distortion in a received signal, the second device 104 may perform the same operations performed by the first device 102 except for those operations that are useful to examine the depth or location of an implanted second device 104. Thus, examples of the logical operations of the second device are discussed below in relation to FIGS. 5-14. The processor 402 may be of various forms such as a general purpose programmable processor, application specific processor, hardwired digital logic, and/or various combinations.

The communication circuitry 404 includes both a transmitter circuit 406 and a receiver circuit 408 for sending and receiving wireless signals. This allows the processor 402 to receive information such as programming, stimulation parameters and the like from the external device 114. This also allows the processor 402 to send information such as sensed physiological data to the external device 114, including sending such information in real-time as the physiological signals are being sensed.

The stimulation circuitry 410 in the example shown allows the implantable medical device 102 to interact with the body of the patient 110. The stimulation circuitry 410 may produce a stimulation signal that includes stimulation pulses of a given amplitude, such as a given electrical current amplitude. In accordance with the stimulation therapy algorithm being performed by the processor 402, the stimulation circuitry 410 may alter the stimulation amplitude of the stimulation therapy as requested by the processor 402 to provide effective stimulation therapy, such as effective adaptive deep brain stimulation.

One manner of providing effective stimulation therapy utilizes feedback in the form of sensed physiological signals. Sensing circuitry 412 senses these physiological signals such as local field potential signals and provides the sensed signal to the processor 402. For instance, with adaptive deep brain stimulation, the sensing circuitry 412 may be used to sense local field potential signals during the ongoing application of stimulation signals. The sensed local field potential signals are analyzed by the processor 402 in order to then request stimulation amplitude changes by the stimulation circuitry 410. The processor 402 compares the sensed local field potential signal power to the physiological thresholds to determine whether to alter the stimulation amplitude.

The physiological thresholds and stimulation amplitude limits that are used by the processor 402 when providing adaptive stimulation therapy may be established in various ways. For instance, the processor 402 may perform an auto-capture algorithm to determine what thresholds and stimulation amplitude limits are appropriate for the patient at that time. However, manual adjustment of these automatically captured values may be required for certain patients. Embodiments of the external device 114 may therefore receive data from the implantable medical device 102 during the auto-capture and display this data for review by the clinician or other user. Such a 500 display is shown in FIG. 5 where the raw data being obtained by the processor 402 of the implantable medical device 102 can be seen by the user.

In FIG. 5, the display 500 includes the local field potential signal 502 as a measure of sensed power that occurs while stimulation is provided at a minimum stimulation amplitude established for the patient over a timeline 552. It will be appreciated that while the local field potential signal 502 is presented as a time domain signal as shown in FIG. 5, the local field potential signal 502 may be presented in other manners, such as in a frequency domain, or in other quantitative representations that are useful to the clinician. The display 500 also includes the local field potential signal 504 as a measure of sensed power that occurs while stimulation is provided at a maximum stimulation amplitude established for the patient over a timeline 554. The display 500 further includes the display of the horizontal line representing the upper threshold 510 and the horizontal line representing the lower threshold 506 that have been automatically captured by the implantable medical device 102. Thus, the clinician or other user of the external device 114 can see the local field potential signal and the resulting automatically captured thresholds for the specified stimulation amplitude limits and determine whether an adjustment to any parameter should be made. As the displayed signals 502, 504 are static on the display, the clinician is able to make adjustments while continuing to view and be guided by the static displayed signals 502, 504. To further distinguish the two signals 502, 504, a first color may be used for signal 502 while a second color may be used for signal 504 on the display. It will be appreciated that the displays of the local field potential data as in FIG. 5 may auto-scale the vertical axis to include the threshold values.

To make such adjustments, the horizontal lines 510, 506 representing the upper and lower thresholds respectively may be associated with corresponding user controls 514, 512. User control 514 is a slider that allows the user to slide the control 514 up and down to adjust the upper threshold higher or lower. User control 512 is a slider that allows the user to slide the control 512 up and down to adjust the lower threshold higher or lower. As shown in FIG. 5, the user has moved both slider 514 and slider 512 upward to raise both the upper threshold to a setting of 40 and the lower threshold to a setting of 37. The auto-captured upper and lower thresholds remain displayed as a dashed lines 510, 506 respectively while the resulting manual upper and resulting manual lower thresholds are shown as solid horizontal lines 524, 516, respectively. Further, in an advanced view, the value of the old upper threshold 510, new upper threshold 526, old the lower threshold 508, and new lower threshold 518 are displayed alongside corresponding up/down button pairs 536/538 and 532/534. The up/down button pairs allow the upper and lower thresholds to be set in small increments by a single click of the up or down button of the pair which provides more precise control of the thresholds relative to the sliders 514, 512. It can be seen that the auto-captured upper threshold value was “34” and has been manually adjusted upward to a value of “40” while the auto-captured lower threshold value was “28” and has been adjusted upward to a value of “37.”

While FIG. 5 shows an upper threshold and lower threshold and upper threshold controls and lower threshold controls which provide a dual threshold implementation, it will be appreciated that the device may implement a single threshold mode of operation. In that case, a single threshold may be displayed along with a single threshold control.

In addition to the thresholds, the display 500 of FIG. 5 also provides information and controls to the clinician for the stimulation amplitude limits. In the advanced view, the lower stimulation amplitude limit 530 used during auto-capture is shown alongside the upper threshold values while the upper stimulation amplitude limit 528 is shown alongside the lower threshold values. Likewise, a stimulation amplitude scale 550 is shown with a stimulation amplitude maximum limit control 540 and a stimulation amplitude maximum limit control 542. In this example, the controls 540 and 542 are sliders that the clinician may manually move to set the stimulation amplitude limits.

Upon activating stimulation that will be controlled according to the thresholds, the current stimulation level being applied may be controlled by the clinician. A control 548, such as a slider, is present to provide user control of the stimulation amplitude to be provided when the stimulation amplitude is not automatically adapted by the therapy control algorithm. For the stimulation amplitude currently being provided via control 548, a demonstration of the reach of the current at that amplitude is shown at the graphical representation 522. An up/down button pair 544/546 is also displayed as a control in this example to allow the clinician to make more precise adjustments than the slider 548 to the stimulation amplitude currently being provided when the stimulation amplitude is not automatically adapted by the therapy control algorithm. In the case of either the slider 548 or the up/down button pair 544/546, moving the control to set the stimulation amplitude beyond either stimulation amplitude limit may shift the stimulation amplitude limits accordingly.

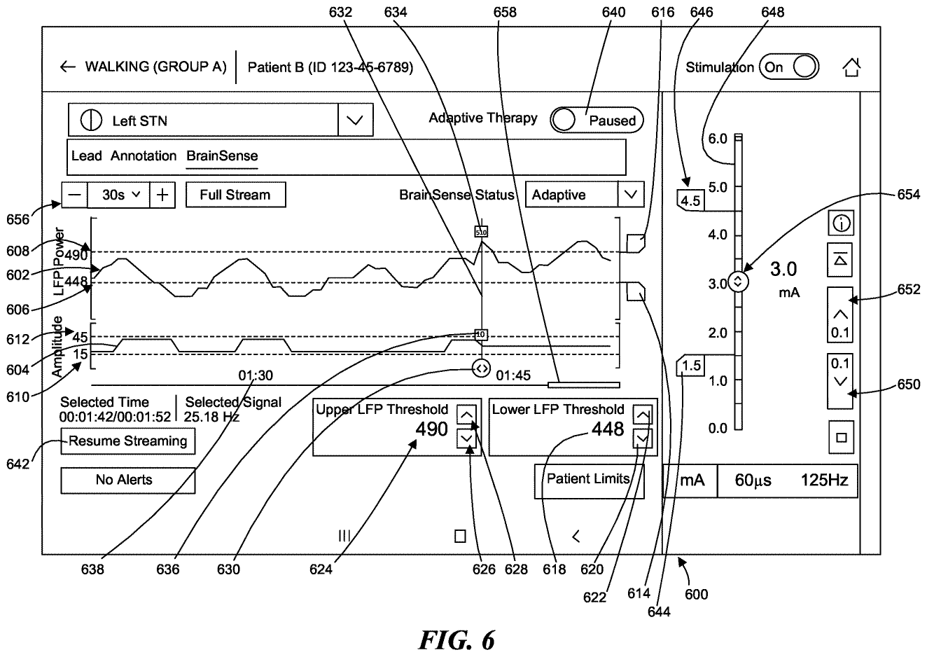

Upon reviewing the auto-capture thresholds and manually adjusting them as necessary, the use of those settings during stimulation therapy can then be viewed at the external device 114 in the display 600 shown in FIG. 6. Here, the sensed local field potential signal 602 may be displayed in real-time as the data is being streamed from the implantable medical device 102 to the external device 114 in real-time. The thresholds 606, 608 currently being used may also be displayed as horizontal lines together with the local field potential signal 602. It will again be appreciated that while the local field potential signal 602 is presented as a time domain signal as shown in FIG. 6, the local field potential signal 602 may be presented in other manners, such as in a frequency domain, or in other quantitative representations that are useful to the clinician. It will also be appreciated that the displays of the local field potential data may auto-scale the vertical axis to include the threshold values.

Likewise, the stimulation signal 604 that the implantable medical device 102 is generating to maintain the local field potentials within the thresholds is also shown in real-time and on a common time axis 638 together with and adjacent to the local field potential signal 602. This allows the clinician to easily see the relationship in real-time between the local field potential signal being sensed and the stimulation signal being applied. The minimum stimulation amplitude limit 610 and maximum stimulation amplitude limit 612 are also shown together with the stimulation signal as horizontal lines. It should be appreciated that the stimulation signal 604 as shown in FIG. 6 is a plot of the stimulation amplitude as a function of time to represent the actual stimulation that is a series of charge-balanced biphasic pulses. Alternatively, the stimulation signal 602 could be shown as the pulses if on an appropriate time scale, but for purposes of viewing the change in amplitude over time, the plot of the stimulation amplitude is effective.

The clinician may wish to again change the thresholds and/or the stimulation amplitude limits. This can be done in real-time by using controls like those discussed for the auto-capture display 500 of FIG. 5. In the real-time stimulation therapy display 600 of FIG. 6, there are threshold controls 616, 614, such as sliders, that allow the user to manually control the upper threshold and the lower threshold higher or lower. An up/down button pair 628/626 may also be displayed to allow a user to make finer adjustments to the upper threshold, while an up/down button pair 622/620 may also be displayed to allow a user to make finer adjustments to the lower threshold. The value of the upper threshold 624 and lower threshold 618 may also be displayed to further assist in making the finer adjustments. In the example show, the upper threshold currently has a value of “490” while the lower threshold currently has a value of “448.” It will be appreciated that these thresholds may vary substantially from patient to patient, as can be seen when comparing the values of FIG. 5 to those of FIG. 6, due to various factors such as disease state, medication state, proximity of the electrodes to the target area, and the like.

While FIG. 6 shows an upper threshold and lower threshold and upper threshold controls and lower threshold controls which provide a dual threshold implementation, it will be appreciated that the device may implement a single threshold mode of operation. In that case, a single threshold may be displayed along with a single threshold control.

The display 600 also includes controls 646, 644, such as sliders, for adjusting the maximum stimulation amplitude limit and the minimum stimulation amplitude limit in real-time on a stimulation amplitude scale 648. During stimulation that will be controlled according to the thresholds, the current stimulation level being applied may be controlled by the clinician. A control 654, such as a slider, is present to provide user control of the stimulation amplitude to be provided when the stimulation amplitude is not automatically adapted by the therapy control algorithm. An up/down button pair 652/650 is also displayed as a control in this example to allow the clinician to make more precise adjustments than the slider 654 to the stimulation amplitude currently being provided when the stimulation amplitude is not automatically adapted by the therapy control algorithm. In the case of either the slider 654 or the up/down button pair 652/650, moving the control to set the stimulation amplitude beyond either stimulation amplitude limit may shift the stimulation amplitude limits accordingly.

In the example shown, the common time axis being displayed spans 30 seconds, which can be set via a time axis control 656, such as a drop down menu of options. Furthermore, the clinician may also choose to pause the streaming display of the local field potential signal using a control button 642 to pause and resume so that the local field potential signal and stimulation signal that span of the specified amount of time persist on the display. Likewise, the clinician may choose to pause the adaptive stimulation therapy being applied by the implantable medical device 102 by using a control 640 such as a slider or button.

With the streaming display of data being paused so that the local field potential signal and the stimulation signal persist on the display, the clinician may then adjust a visual indicator 632, shown as a vertical line, to move along the common time axis 638 by user manipulation of a timeline slide control 630 where the visual indicator 632 intersects both the stimulation signal 604 and the local field potential signal 602. The visual indicator 632 may further include a stimulation amplitude indicator 636 to specify the stimulation amplitude being applied at that moment in time and may also further include a local field potential power indicator 634 to specify the local field potential power at that same point in time. This is useful so the clinician can analyze a particular point of interest such as a point in time when the local field potential signal has exceeded a threshold. To further assist in viewing the waveform, a control 658 may be included to allow the user to move the common time axis to show other portions of the local field potential signal and stimulation signal from the greater region of time that has been recorded, such as to view other regions of time from the past 5 minutes.

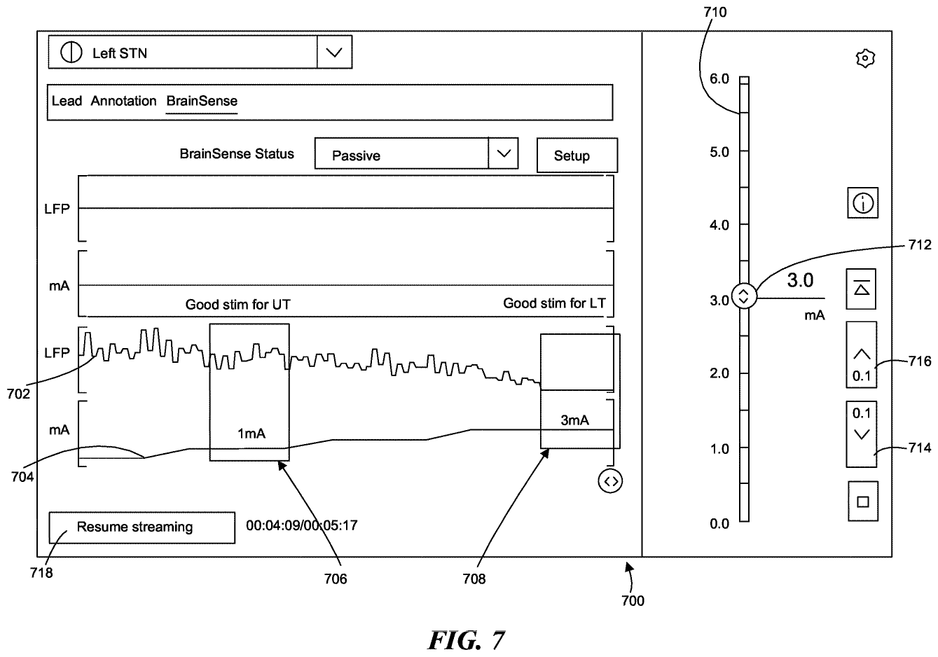

FIG. 7 shows another display 700 that may be provided by the external device 114 to allow the clinician to add annotations for the local field potential signal 702 and corresponding stimulation signal 704. It will be again appreciated that while the local field potential signal 702 is presented as a time domain signal as shown in FIG. 7, the local field potential signal 702 may be presented in other manners, such as in a frequency domain, or in other quantitative representations that are useful to the clinician. It should also be appreciated again that the stimulation signal 704 as shown in FIG. 7 is a plot of the stimulation amplitude as a function of time to represent the actual stimulation that is a series of charge-balanced biphasic pulses. Alternatively, the stimulation signal 704 could be shown as the pulses if on an appropriate time scale, but for purposes of viewing the change in amplitude over time, the plot of the stimulation amplitude is effective. In a first annotation 706, the clinician has boxed an area believed to show that there has been good stimulation therapy for addressing the local field potential in proximity to the upper threshold. In a second annotation 708, the clinician has boxed an area believed to show that there has been good stimulation therapy for addressing the local field potential in proximity to the lower threshold. The clinician may select a pause/resume button 718 to allow the real-time display of the signals to be paused to more easily add the annotations 706, 708 and then select the pause/resume button to resume the real-time streaming. In this example, the display 700 includes various other aspects like that of the display 600 in FIG. 6, such as a stimulation amplitude scale 710, a stimulation amplitude slider 712, and an up/down button pair 716/714 to allow for more precise adjustment of the stimulation amplitude.

FIG. 8 shows an example of logical operations that may be performed by the external device 114 to generate the automatic threshold capture display like that of FIG. 5 and receive the control inputs by the user. In this example, the external device 114 begins by instructing the implantable medical device 102 to begin the automatic threshold capture either within specified or otherwise calculated upper and lower amplitude limits at an operation 802. The external device 114 then receives data representative of the sensed local field potential signals that have occurred during the auto-capture process at an operation 804. The external device 114 is then ready to display the information shown above in the display 500 of FIG. 5.

The external device 114 then produces the display 500 by contemporaneously displaying the various aspects. For instance, the external device displays horizontal threshold lines found by the auto-capture algorithm including the upper and lower values at an operation 806. The external device 114 displays the sensed local field potential signals at an operation 808. The external device 114 displays the manual threshold controls such as sliders and buttons as well as the horizontal threshold lines, if any, that are manually set along with corresponding threshold numerical values at an operation 810. The external device 114 also displays the manual stimulation amplitude limit controls and values at an operation 812.

The external device 114 is also monitoring for user input via the user controls while providing the display 500. For instance, the external device is monitoring for user input at the threshold controls to determine if the user has manipulated the control at an operation 814. If so, then the external device 114 updates the display with the resulting threshold and sends an instruction to the implantable medical device 102 to implement the new threshold at an operation 818. The external device 114 continues to monitor for additional user manipulation of the threshold controls.

Likewise, the external device 114 is monitoring for user input at the stimulation amplitude controls to determine if the user has manipulated the control at an operation 822. If so, then the external device 114 updates the display with the resulting amplitude limit setting and sends an instruction to the implantable medical device 102 to implement the new amplitude limit at an operation 824. The external device 114 continues to monitor for additional user manipulation of the amplitude limit controls.

The external device 114 also monitors for the user to select to save and exit from the auto-capture display 500 at an operation 816. Until the user selects to save and exit, the external device 114 continues to display the various items via the operations 806, 808, 810, and 812 and continues to monitor for user manipulation of controls via the operations 814 and 822.

FIG. 9 shows an example of logical operations that may be performed by the external device 114 to generate the real-time stimulation therapy display like that of FIG. 6 and receive control inputs by the user. In this example, the external device 114 begins by instructing the implantable medical device 102 to begin the adaptive stimulation therapy with the previously established thresholds and stimulation amplitude limits at an operation 902. This instruction to perform adaptive stimulation therapy involves the implantable medical device 102 applying a stimulation signal within a range of amplitudes specified by the amplitude limits while sensing the local field potential signal and comparing the signal level to the upper and lower thresholds in order to adapt the stimulation signal. The external device 114 also instructs the implantable medical device to stream the sensed local field potential signal and stimulation signal in real-time to the external device 114. The external device 114 then receives data representative of the sensed local field potential signals and corresponding stimulation signals at an operation 904. The external device 114 is then ready to display the information shown above in the display 600 of FIG. 6 and display 700 of FIG. 7.

The external device 114 then produces the display 600 and/or 700 by contemporaneously displaying the various aspects. For instance, the external device 114 displays the local field potential signal and the stimulation signal in real-time along the common time axis at an operation 906. The external device 114 displays the horizontal threshold lines that represent the thresholds being used for the stimulation therapy including the upper and lower values and that can be manually adjusted at an operation 922. Here the external device 114 also displays the manual threshold controls such as sliders and buttons along with corresponding threshold numerical values. The external device 114 also displays the manual stimulation amplitude limit controls and values at an operation 928. Additionally, the external device 114 displays the timeline with visual indicator and related values for the local field potential and the stimulation amplitude for the point in time where the visual indicator is located at an operation 934.

The external device 114 is also monitoring for user input via the user controls while providing the display 600 and/or 700. For instance, the external device is monitoring for user input at the pause/resume control to determine if the user has selected to pause the real-time display of the signals at an operation 908. If pause is selected, then the external device 114 maintains the display of the current time axis data at an operation 910 which allows the clinician to carefully review the relationship of the local field potential signal, the thresholds, and the application of stimulation between the amplitude limits for that period of time. The external device monitors for whether the user has selected to resume streaming of the real-time data at an operation 912.

The external device 114 also monitors whether the user selects to add an annotation to the persistently displayed signals during the pause of the real-time streaming like that shown in display 700 of FIG. 7. If there has been a selection to add an annotation, then the external device 114 receives the annotation as user input and displays the annotation at an operation 916.

The external device 114 monitors the threshold controls to determine if the user has manipulated the control at an operation 924. If there has been manipulation of the controls, then the external device 114 updates the display with the resulting threshold and sends an instruction to the implantable medical device 102 to implement the new threshold in real-time for the ongoing stimulation therapy at an operation 926. The external device 114 continues to monitor for additional user manipulation of the threshold controls.

Likewise, the external device 114 monitors for user input at the stimulation amplitude controls to determine if the user has manipulated the control at an operation 930. If there has been manipulation of the controls, then the external device 114 updates the display with the resulting amplitude limit setting and sends an instruction to the implantable medical device 102 to implement the new amplitude limit in real-time for the ongoing stimulation therapy at an operation 932. The external device 114 continues to monitor for additional user manipulation of the amplitude limit controls.

As an additional feature of this example, the external device 114 monitors for user input at the visual indicator control and displayed timeline control at an operation 936. If there has been manipulation of either control, then the external device 114 updates the displayed visual indicator or timeline period as well as the numerical values of the local field potential signals and stimulation signals for the point in time where the visual indicator is located at an operation 938.

The external device 114 also monitors for the user to select to save and exit from the real-time stimulation therapy display 600 and/or 700 at an operation 918. Until the user selects to save and exit, the external device 114 continues to display the various items via the operations 906, 922, 928, and 934 and continues to monitor for user manipulation of controls via the operations 908, 912, 914, 924, 930, and 936.

As can be appreciated from the preceding discussion, embodiments disclosed herein provide a user of the external device 114 such as a clinician the ability to view raw physiological signal information obtained during the auto-capture function of the implantable medical device to see the relationship of the local field potential signal and the auto-captured thresholds, enabling the clinician to make informed decisions regarding adjusting the thresholds and the stimulation amplitudes. Furthermore, it can be appreciated that embodiments disclosed herein further provide a user of the external device the ability to monitor in real-time the stimulation therapy and specifically the relationship of the local field potential signal and the stimulation signal, enabling the clinician to make informed decisions regarding adjusting the thresholds and stimulation amplitudes and to enter annotations regarding the visual assessment.

It will be further appreciated that the display of the local field potential signal, threshold(s), and threshold control(s) as shown and described above can be provided in a passive sensing context where the implantable device is sensing the local field potential but not automatically adapting stimulation. The thresholds may be used for other purposes including alerting, data recording, and the like. In this context the thresholds may still be manually controlled by a clinician as discussed above. At least one local field potential threshold can be set to identify the desired range of local field potential levels, with one threshold representing no or acceptable symptoms and the other threshold indicating no side effects. In an exemplary scenario, a patient will go home from an office visit and the implantable device will continue recording local field potential signals until the next follow up session. At the next follow up session, the local field potential signal is plotted relative to these thresholds, and these thresholds are also used to categorize the local field potential data relative to them. The local field potential data can be viewed as above/below/between these thresholds. As noted above, the displays of the local field potential data may auto-scale the vertical axis to include the threshold values.

The invention may further be described as embodiments that provide systems, devices, and methods to provide adaptive neurostimulation therapy from an implantable neurostimulation device by use of an external device. This includes the external device including a processor sending an instruction using communication circuitry of the external device to the implantable neurostimulation device to perform an automatic threshold capture for providing neurostimulation therapy while sensing a local field potential signal. This also includes the processor of the external device receiving through the communication circuitry data representative of the sensed local field potential signal occurring during automatic threshold capture from the implantable neurostimulation device and the external device having a visual display that displays the sensed local field potential signal.

The invention may further be described as embodiments provide systems, devices, and methods to provide adaptive neurostimulation therapy from an implantable neurostimulation device by use of an external device. This includes the external device including a processor sending an instruction using communication circuitry of the external device to the implantable neurostimulation device to provide adaptive stimulation therapy by applying a stimulation signal within a range of first and second amplitudes while sensing a local field potential signal and comparing the local field potential signal to at least one threshold. This includes the processor of the external device receiving through the communication circuitry streaming data representative of the sensed local field potential signal from the implantable neurostimulation device and displaying the sensed local field potential signal represented by the streaming data at the external device. This also includes the external device having a visual display such that the processor causes the visual display to display at least one threshold with the sensed local field potential signal and displaying at least one threshold control. Upon receiving a manipulation of at least one threshold control by a user of the external device, this further includes the processor causing the visual display to display at least one resulting threshold with the sensed field potential and sending by the processor using the communication circuitry an instruction to the implantable neurological device to implement the resulting at least one threshold in real-time while providing the adaptive stimulation therapy.

The invention may further be described as embodiments provide systems, devices, and methods to provide adaptive neurostimulation therapy from an implantable neurostimulation device by use of an external device. This includes the external device having a processor that uses communication circuitry of the external device to send an instruction to the implantable neurostimulation device to provide adaptive stimulation therapy by applying a stimulation signal within a range of first and second amplitudes while sensing a local field potential signal and comparing the local field potential signal to at least one threshold. This includes the processor using the communication circuitry to receive at streaming data representative of the sensed local field potential signal from the implantable neurostimulation device and the processor uses a visual display of the external device to display the sensed local field potential represented by the streaming data. This also includes the processor using the communication circuitry to receive streaming data representative of the stimulation signal from the implantable neurostimulation device and the processor uses a visual display of the external device to display the stimulation signal adjacent the sensed local field potential signal along a common time axis. This further includes the processor using the visual display to provide a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

The following are illustrative of the techniques described herein:

Example 1: A method of providing adaptive neurostimulation therapy from an implantable neurostimulation device by use of an external device, comprising: sending an instruction by the external device to the implantable neurostimulation device to perform an automatic threshold capture for providing neurostimulation therapy while sensing a local field potential signal; receiving at the external device data representative of the sensed local field potential signal occurring during automatic threshold capture from the implantable neurostimulation device; and displaying the sensed local field potential signal at the external device.

Example 2: The method of example 1, further comprising: displaying an auto-captured upper threshold and an auto-captured lower threshold with the sensed local field potential.

Example 3: The method of example 2, wherein displaying the sensed local field potential signal comprises displaying the local field potential signal when the auto-capture function utilizes a stimulation signal at a lower stimulation amplitude limit adjacent to displaying the local field potential signal when the auto-capture function utilizes a stimulation signal at an upper stimulation amplitude limit.

Example 4: The method of example 3, further comprising: displaying a manual upper threshold and a manual lower threshold with the sensed local field potential; displaying an upper threshold control and a lower threshold control; and upon receiving a manipulation of the upper threshold control or the lower threshold control by a user of the external device, displaying at the external device a resulting upper threshold or resulting lower threshold with the sensed field potential; and sending by the external device an instruction to the implantable neurological device to implement the resulting upper threshold or resulting lower threshold when providing stimulation therapy.

Example 5: The method of example 4, wherein the upper threshold control comprises a first threshold slider and wherein the lower threshold control comprises a second threshold slider.

Example 6: The method of example 4, wherein the upper threshold control comprises a first threshold button pair and wherein the lower threshold control comprises a second threshold button pair.

Example 7: The method of example 1, further comprising: displaying an upper stimulation amplitude limit control and a lower amplitude limit control; and upon receiving a manipulation of the upper amplitude limit control or the lower amplitude limit control by the user of the external device, displaying at the external device a resulting upper amplitude limit or resulting lower amplitude limit and sending by the external device an instruction to the implantable neurological device to implement the resulting upper amplitude limit or resulting lower amplitude limit when providing stimulation therapy.

Example 8: The method of example 7, wherein the upper amplitude limit control comprises a first limit slider and wherein the lower amplitude limit control comprises a second limit slider.

Example 9: The method of example 7, further comprising a stimulation amplitude button pair.

Example 10: The method of example 3, wherein displaying the local field potential signal further comprises displaying the local field potential signal when the auto-capture function utilizes a stimulation signal at the lower stimulation amplitude limit in a first color and displaying the local field potential signal when the auto-capture function utilizes a stimulation signal at the upper stimulation amplitude limit in a second color.

Example 11: A method of providing adaptive neurostimulation therapy from an implantable neurostimulation device by use of an external device, comprising: sending an instruction by the external device to the implantable neurostimulation device to provide adaptive stimulation therapy by applying a stimulation signal within a range of first and second amplitudes while sensing a local field potential signal and comparing the local field potential signal to at least one threshold; receiving at the external device streaming data representative of the sensed local field potential signal from the implantable neurostimulation device; displaying the sensed local field potential signal represented by the streaming data at the external device; displaying at least one threshold with the sensed local field potential signal; displaying at least one threshold control; and upon receiving a manipulation of the at least one threshold control by a user of the external device, displaying at the external device a resulting at least one threshold with the sensed field potential and sending by the external device an instruction to the implantable neurological device to implement the resulting at least one threshold in real-time while providing the adaptive stimulation therapy.

Example 12: The method of example 11, further comprising receiving at the external device streaming data representative of the stimulation signal from the implantable neurostimulation device; an displaying the stimulation signal adjacent the sensed local field potential at the external device along a common time axis.

Example 13: The method of example 12, further comprising providing a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

Example 14: The method of example 11, wherein the at least one threshold control comprises an upper threshold control and a lower threshold control and wherein the upper threshold control comprises a first threshold slider and wherein the lower threshold control comprises a second threshold slider.

Example 15: The method of example 11, wherein the at least one threshold control comprises an upper threshold control and a lower threshold control and wherein the upper threshold control comprises a first threshold button pair and wherein the lower threshold control comprises a second threshold button pair.

Example 16: The method of example 11, further comprising: displaying an upper amplitude limit control and a lower amplitude limit control; and upon receiving a manipulation of the upper amplitude limit control or the lower amplitude limit control by the user of the external device, displaying at the external device a resulting upper amplitude limit or resulting lower amplitude limit and sending by the external device an instruction to the implantable neurological device to implement the resulting upper amplitude limit or resulting lower amplitude limit when providing stimulation therapy.

Example 17: The method of example 16, further comprising a stimulation amplitude button pair.

Example 18: The method of example 11, further comprising receiving user input to create an annotation in proximity to a portion of the display of the local field potential signal and displaying the annotation in proximity to the portion of the display of the local field potential signal.

Example 19: A method of providing adaptive neurostimulation therapy from an implantable neurostimulation device by use of an external device, comprising: sending an instruction by the external device to the implantable neurostimulation device to provide adaptive stimulation therapy by applying a stimulation signal within a range of first and second amplitudes while sensing a local field potential signal and comparing the local field potential signal to at least one threshold; receiving at the external device streaming data representative of the sensed local field potential signal from the implantable neurostimulation device; displaying the sensed local field potential represented by the streaming data at the external device receiving at the external device streaming data representative of the stimulation signal from the implantable neurostimulation device; displaying by the external device the stimulation signal adjacent the sensed local field potential signal at the external device along a common time axis; and providing by the external device a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

Example 20: The method of example 19, further comprising: displaying an upper threshold and a lower threshold with the sensed local field potential; displaying an upper threshold control and a lower threshold control; and upon receiving a manipulation of the upper threshold control or the lower threshold control by a user of the external device, displaying at the external device a resulting upper threshold or resulting lower threshold with the sensed field potential and sending by the external device an instruction to the implantable neurological device to implement the resulting upper threshold or resulting lower threshold in real-time while providing the adaptive stimulation therapy.

While embodiments have been particularly shown and described, it will be understood by those skilled in the art that various other changes in the form and details may be made therein without departing from the spirit and scope of the invention.

1-15. (canceled)

16. A method of providing adaptive neurostimulation therapy from an implantable neurostimulation device by use of an external device, comprising:
sending an instruction by the external device to the implantable neurostimulation device to perform an automatic threshold capture for providing neurostimulation therapy while sensing a local field potential signal;
receiving at the external device data representative of the sensed local field potential signal occurring during automatic threshold capture from the implantable neurostimulation device; and
displaying the sensed local field potential signal at the external device.

17. The method of claim 16, further comprising:
displaying an auto-captured upper threshold and an auto-captured lower threshold with the sensed local field potential.

18. The method of claim 17, wherein displaying the sensed local field potential signal comprises displaying the local field potential signal when the auto-capture function utilizes a stimulation signal at a lower stimulation amplitude limit adjacent to displaying the local field potential signal when the auto-capture function utilizes a stimulation signal at an upper stimulation amplitude limit.

19. The method of claim 18, further comprising:
displaying a manual upper threshold and a manual lower threshold with the sensed local field potential;
displaying an upper threshold control and a lower threshold control; and
upon receiving a manipulation of the upper threshold control or the lower threshold control by a user of the external device, displaying at the external device a resulting upper threshold or resulting lower threshold with the sensed field potential; and
sending by the external device an instruction to the implantable neurological device to implement the resulting upper threshold or resulting lower threshold when providing stimulation therapy.

20. The method of claim 19, wherein the upper threshold control comprises a first threshold slider and wherein the lower threshold control comprises a second threshold slider.

21. The method of claim 19, wherein the upper threshold control comprises a first threshold button pair and wherein the lower threshold control comprises a second threshold button pair.

22. The method of claim 16, further comprising:
displaying an upper stimulation amplitude limit control and a lower amplitude limit control; and
upon receiving a manipulation of the upper amplitude limit control or the lower amplitude limit control by the user of the external device, displaying at the external device a resulting upper amplitude limit or resulting lower amplitude limit and sending by the external device an instruction to the implantable neurological device to implement the resulting upper amplitude limit or resulting lower amplitude limit when providing stimulation therapy.

23. The method of claim 22, wherein the upper amplitude limit control comprises a first limit slider and wherein the lower amplitude limit control comprises a second limit slider.

24. The method of claim 22, further comprising a stimulation amplitude button pair.

25. The method of claim 18, wherein displaying the local field potential signal further comprises displaying the local field potential signal when the auto-capture function utilizes a stimulation signal at the lower stimulation amplitude limit in a first color and displaying the local field potential signal when the auto-capture function utilizes a stimulation signal at the upper stimulation amplitude limit in a second color.

26. A method of providing adaptive neurostimulation therapy from an implantable neurostimulation device by use of an external device, comprising:
sending an instruction by the external device to the implantable neurostimulation device to provide adaptive stimulation therapy by applying a stimulation signal within a range of first and second amplitudes while sensing a local field potential signal and comparing the local field potential signal to at least one threshold;
receiving at the external device streaming data representative of the sensed local field potential signal from the implantable neurostimulation device;
displaying the sensed local field potential signal represented by the streaming data at the external device;
displaying at least one threshold with the sensed local field potential signal;
displaying at least one threshold control; and
upon receiving a manipulation of the at least one threshold control by a user of the external device, displaying at the external device a resulting at least one threshold with the sensed field potential and sending by the external device an instruction to the implantable neurological device to implement the resulting at least one threshold in real-time while providing the adaptive stimulation therapy.

27. The method of claim 26, further comprising:
receiving at the external device streaming data representative of the stimulation signal from the implantable neurostimulation device; and
displaying the stimulation signal adjacent the sensed local field potential at the external device along a common time axis.

28. The method of claim 27, further comprising providing a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

29. The method of claim 26, wherein the at least one threshold control comprises an upper threshold control and a lower threshold control and wherein the upper threshold control comprises a first threshold slider and wherein the lower threshold control comprises a second threshold slider.

30. The method of claim 26, wherein the at least one threshold control comprises an upper threshold control and a lower threshold control and wherein the upper threshold control comprises a first threshold button pair and wherein the lower threshold control comprises a second threshold button pair.

31. The method of claim 26, further comprising:
displaying an upper amplitude limit control and a lower amplitude limit control; and
upon receiving a manipulation of the upper amplitude limit control or the lower amplitude limit control by the user of the external device, displaying at the external device a resulting upper amplitude limit or resulting lower amplitude limit and sending by the external device an instruction to the implantable neurological device to implement the resulting upper amplitude limit or resulting lower amplitude limit when providing stimulation therapy.

32. The method of claim 31, further comprising a stimulation amplitude button pair.

33. The method of claim 26, further comprising receiving user input to create an annotation in proximity to a portion of the display of the local field potential signal and displaying the annotation in proximity to the portion of the display of the local field potential signal.

34. A method of providing adaptive neurostimulation therapy from an implantable neurostimulation device by use of an external device, comprising:
sending an instruction by the external device to the implantable neurostimulation device to provide adaptive stimulation therapy by applying a stimulation signal within a range of first and second amplitudes while sensing a local field potential signal and comparing the local field potential signal to at least one threshold;
receiving at the external device streaming data representative of the sensed local field potential signal from the implantable neurostimulation device;
displaying the sensed local field potential represented by the streaming data at the external device;
receiving at the external device streaming data representative of the stimulation signal from the implantable neurostimulation device;
displaying by the external device the stimulation signal adjacent the sensed local field potential signal at the external device along a common time axis; and
providing by the external device a timeline slide control and corresponding visual indicator where the timeline slide control moves the visual indicator along the common time axis and the visual indicator provides a display of a stimulation signal amplitude and a display of a local field potential power at a point in time corresponding to the position of the visual indicator on the common time axis.

35. The method of claim 19, further comprising:
displaying an upper threshold and a lower threshold with the sensed local field potential;
displaying an upper threshold control and a lower threshold control; and
upon receiving a manipulation of the upper threshold control or the lower threshold control by a user of the external device, displaying at the external device a resulting upper threshold or resulting lower threshold with the sensed field potential and sending by the external device an instruction to the implantable neurological device to implement the resulting upper threshold or resulting lower threshold in real-time while providing the adaptive stimulation therapy.
